(19) United States
(12) Patent Application Publication
(10) Pub. No.: US 2023/0419481 A1
(43) Pub. Date: Dec. 28, 2023

(54) IMAGE CAPTURE FOR DIAGNOSTIC TEST RESULTS

(71) Applicant: Exa Health, Inc., Los Altos, CA (US)

(72) Inventors: Kevin J. Miller, Mountain View, CA (US); Mayank Kumar, Sunnyvale, CA (US); Steven Scherf, Oakland, CA (US); Siddarth Satish, Redwood City, CA (US); Thang Sy, Menlo Park, CA (US); Utsha Guha, Fremont, CA (US)

(21) Appl. No.: 18/036,479

(22) PCT Filed: Oct. 26, 2021
(86) PCT No.: PCT/US2021/056670

§ 371 Date: May 11, 2023

Related U.S. Application Data
(60) Provisional application No. 63/115,889, filed on Nov. 19, 2020.

(51) Int. Cl. G06T 7/00; G06V 10/22; G16H 30/40

(52) U.S. Cl. CPC G06T 7/0012; G06V 10/225; G16H 30/40

(57) ABSTRACT

Disclosed is a method of capturing an image of diagnostic test results. The method may include acquiring an image of a diagnostic test positioning card using an image capture device, determining the presence of a plurality of fiducial images on the test positioning card, determining the presence of a diagnostic test result carrier placed on the test positioning card and verifying positioning of the test result carrier on the test positioning card. An image of the test result carrier may then be captured. The method may also include acquiring an image of a test result carrier using an image capture device, determining image features in the image of the test result carrier and verifying positioning of the test result carrier relative to the image capture device using the image features. An image including the test result carrier may then be captured.

### CLAIM OF PRIORITY

This application is a national phase entry of International Application No. PCT/US2021/056670, titled “IMAGE CAPTURE FOR DIAGNOSTIC TEST RESULTS, and filed Oct. 26, 2021, which claims the benefit of the filing date of U.S. Provisional Patent Application Ser. No. 63/115,889, filed Nov. 19, 2020 entitled, “IMAGE CAPTURE FOR DIAGNOSTIC TEST RESULTS,” the entire content of which applications are incorporated herein by reference in their entireties as if explicitly set forth.

### TECHNICAL FIELD

The subject matter disclosed herein generally relates to the technical field of machines and methods that facilitate analysis of test strips, including software-configured variants of such machines and improvements to such variants, and to the technologies by which such machines become improved compared to other machines that facilitate analysis of test strips. Specifically, the present disclosure addresses systems and methods to facilitate image capture of test strips for analysis.

### BACKGROUND

Lateral Flow Assay (LFA) is a type of paper-based platform used to detect the concentration of analyte in a liquid sample. LFA test strips are cost-effective, simple, rapid, and portable tests (e.g., contained within LFA testing devices) that have become popular in biomedicine, agriculture, food science, and environment science, and have attracted considerable interest for their potential to provide rapid diagnostic results directly to patients. LFA-based tests are widely used in hospitals, physicians' offices, and clinical laboratories for qualitative and quantitative detection of specific antigens and antibodies, as well as for products of gene amplification. LFA tests have widespread and growing applications (e.g., in pregnancy tests, malaria tests, COVID-19 antibody tests, COVID-19 antigen tests, or drug tests) and are well-suited for point-of-care (POC) and at-home applications.

### BRIEF DESCRIPTION

To easily identify the discussion of any particular element or act, the most significant digit or digits in a reference number refer to the figure number in which that element is first introduced.

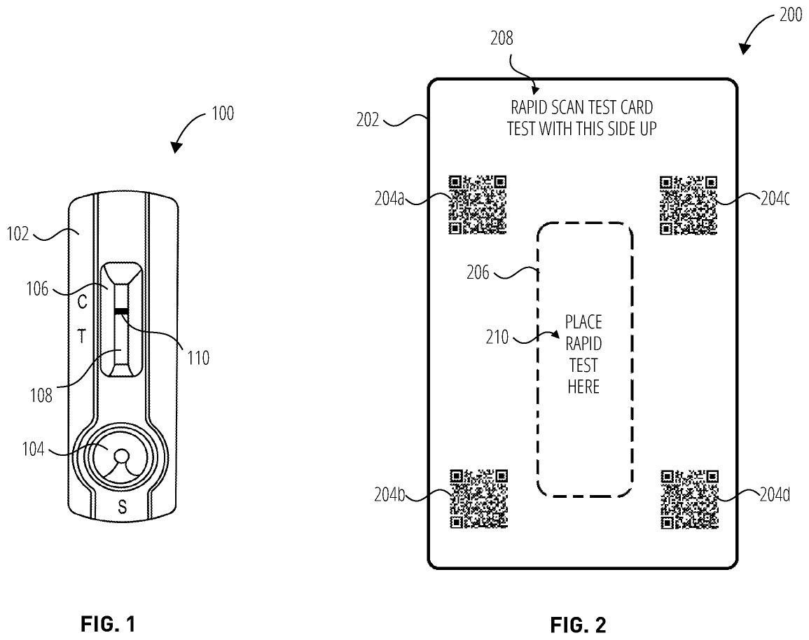

FIG. 1 illustrates a test cassette that may be used in one example of the systems and methods described herein.

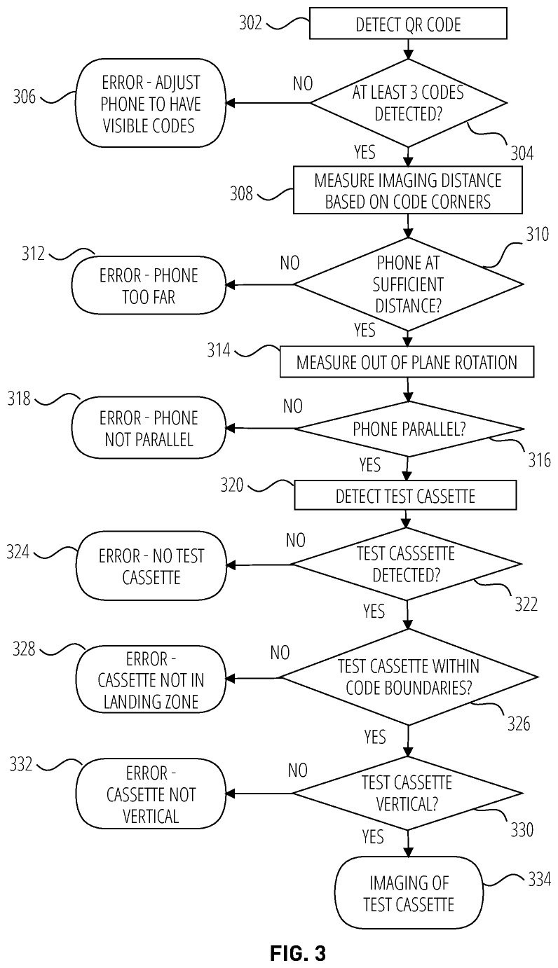

FIG. 2 illustrates a test positioning card that may be used in one example of the systems and methods described herein.

FIG. 3 is a flow chart illustrating a method of capturing an image of a test cassette that is positioned on a test positioning card, in one example.

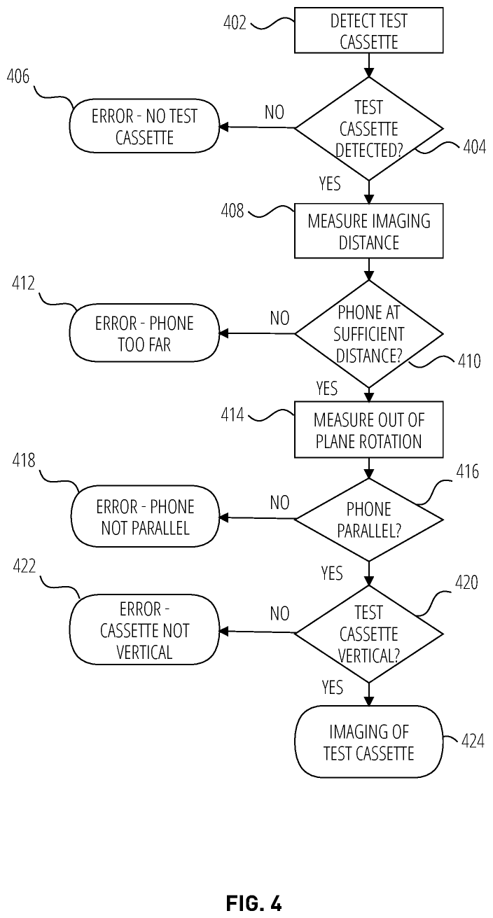

FIG. 4 is a flow chart illustrating a method of capturing an image of a test cassette, in another example.

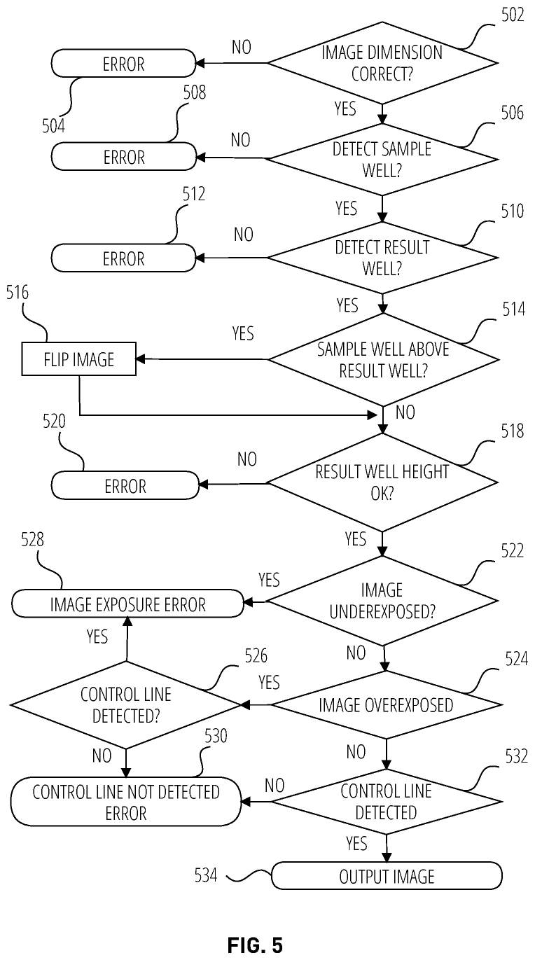

FIG. 5 is a flow chart illustrating a method of verifying an image that has been captured by a computing device using the method described above with reference to FIG. 3 or FIG. 4.

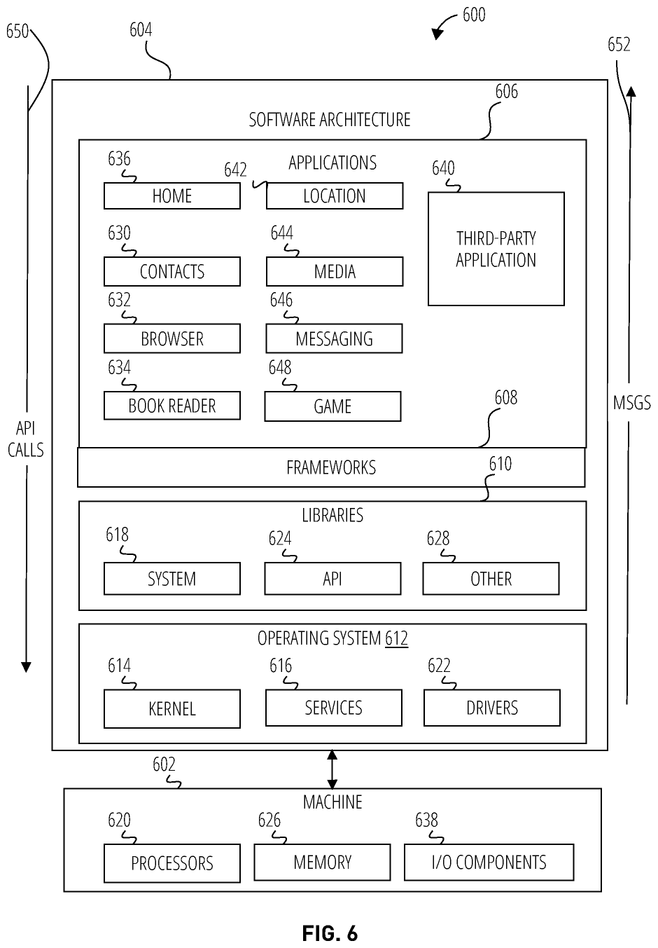

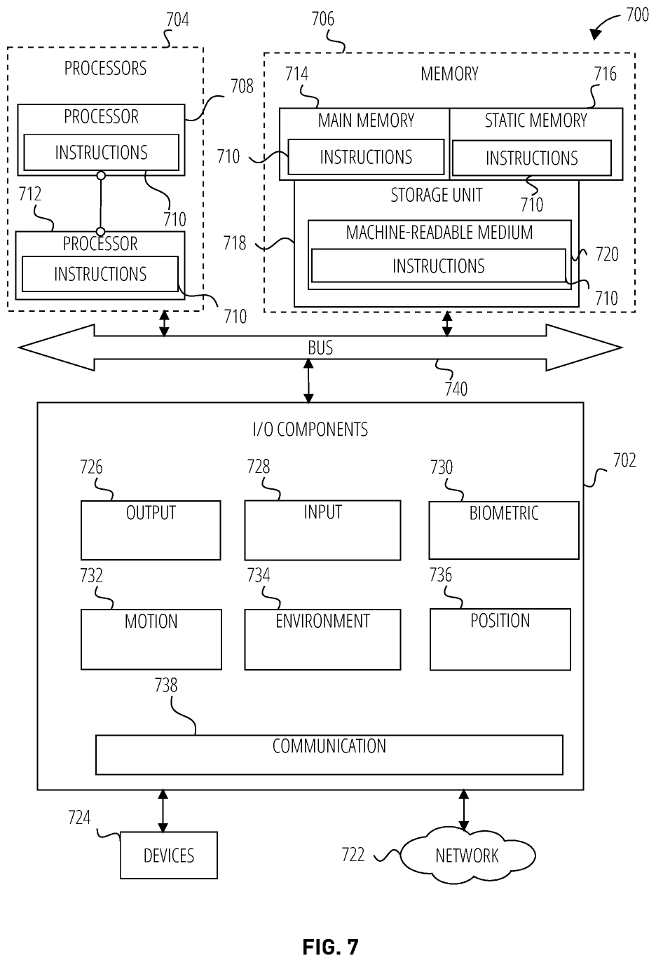

FIG. 6 is block diagram showing a software architecture within which the present disclosure may be implemented, according to an example embodiment.

FIG. 7 is a diagrammatic representation of a machine in the form of a computer system within which a set of instructions may be executed for causing the machine to perform any one or more of the methodologies discussed herein, in accordance with some example embodiments.

### DETAILED DESCRIPTION

Example methods (e.g., algorithms) facilitate the image capture of diagnostic test strips (e.g., LFA test strips), and example systems (e.g., machines configured by special-purpose software) are configured to perform such image capture. Examples merely typify possible variations. Unless explicitly stated otherwise, structures (e.g., structural components, such as modules) are optional and may be combined or subdivided, and operations (e.g., in a procedure, algorithm, or other function) may vary in sequence or be combined or subdivided. In the following description, for purposes of explanation, numerous specific details are set forth to provide a thorough understanding of various example embodiments. It will be evident to one skilled in the art, however, that the present subject matter may be practiced without these specific details.

Diagnostic tests may be performed using LFA test strips, which usually have a designated control line region and a test line region. The diagnostic test strip may be included in a diagnostic test carrier, which may take the form of an LFA test cassette (e.g. test cassette 100 shown in FIG. 1). An LFA test cassette typically has at least one sample well for receiving a sample to be applied to the LFA test strip housed inside the diagnostic test device. Typically, results can be interpreted within 5-30 minutes after putting a sample in the designated sample well of an LFA test cassette. The results, which manifest as visual indicators on the test strip, can be read by a trained healthcare practitioner (HCP) in a qualitative manner, such as by visually determining the presence or absence of a test result line appearing on the LFA test strip.

However, qualitative assessment by a human HCP may be subjective and error prone, particularly for faint lines that are difficult to visually identify. Instead, quantitative assessment of line presence or absence, such as by measuring line intensity or other indicator of line strength, may be more desirable for accurate reading of faint test result lines. Fully or partially quantitative approaches directly quantify the intensity or strength of the test result line or can potentially determine the concentration of the analyte in the sample based on the quantified intensity or other quantified strength of the test result line. Dedicated hardware devices that acquire images of LFA test strips typically include image processing software to perform colorimetric analysis to determine line strength, and often rely upon control of dedicated illumination, exclusion of external lighting, perfect alignment of a built-in camera and the LFA test cassette being imaged, and expensive equipment and software to function properly. More flexible and less expensive approaches may be beneficial.

The methods and systems discussed herein describe a technology for the capture of images of LFA test cassettes that include a diagnostic test result region using standard cameras such as a smartphone camera, which may then automatically be read and analyzed locally or remotely by using computer-based reading and analysis techniques. The method and systems discussed herein allow a user to capture a high quality image of test cassette using standard image capture hardware (such as a commodity phone camera) in the presence of ambient lighting or standard light sources (such as a flash or flashlight adjacent to a phone camera) without the need for dedicated specialized hardware. The methods and system discussed herein allow a lay-person to capture a suitably aligned image of a test cassette using only a smartphone with a camera, with the help of a test positioning card disclosed here.

Once a high-quality and aligned image of a test cassette is acquired, the method and systems described herein also allow the performance of automatic quality control on the acquired test cassette image to ensure that the test strip/result well is well lit, is of sufficiently high resolution, and is properly oriented before performing the analysis of the test strip image to determine test results. If the image quality is not sufficient, the end-user is appropriately warned and prompted to recapture the image.

In one example, once the image has been captured, the test strip is analyzed as described in U.S. Provisional Patent Application No. 63/049,213 filed Jul. 8, 2020 entitled “Neural Network Analysis of LFA Test Strips,” the disclosure of which is incorporated herein as if specifically set forth.

FIG. 1 illustrates a test cassette 100 that may be used in one example of the methods and systems described herein. The test cassette 100, which is one example of a diagnostic test result carrier, includes a housing 102 that has a sample well 104 and a result well 106 defined therein. The result well 106 defines an opening through which a test strip 108 can be viewed and the sample well 104 provides access to one end of the test strip 108. Of course, it will be appreciated that different configurations of presenting diagnostic test results, in different shapes and sizes, with or without a housing, may be utilized herein. The methods and system disclosed herein are equally applicable to wide variations in test cassette design that may house more than one test strip with multiple sample and result wells or with other geometric configurations.

In use of the test cassette 100, a liquid biological sample is placed in the sample well 104 (and thus onto one end of the test strip), which then flows through the housing 102 along the test strip 108, thereby to expose the test strip 108 to the biological sample as is known in the art. Exposure of the test strip 108 to the biological sample will cause one or more visual result markers to appear on the test strip 108 in the result well 106, depending on the nature of the test and the contents of the biological sample. In a typical implementation, the test strip 108 includes a control line 110, which becomes visible when the biological sample reaches it after passing through the test area of the test strip 108, regardless of the contents of the biological sample.

FIG. 2 illustrates a test positioning card 200, according to one example, which is used for aligning a test cassette 100. The test positioning card 200 is printed on a sheet 202 of paper or cardboard and includes a plurality of fiducial markers 204, e.g. fiducial markers 204a to 204d, and a guide outline 206 corresponding generally in shape to the perimeter of the test cassette 100. The test positioning card 200 may also include instructions 208 and instructions 210 to guide a user in the use of the test positioning card 200. In use, a test cassette 100 is placed on the test positioning card 200 to facilitate capture of an image that includes the test strip 108. The image may for example be captured by a smartphone with a camera, but any appropriate computing device (e.g. computing device 700 described below with reference to FIG. 7) may be used.

The fiducial markers 204 in the illustrated example are four QR codes arranged in a rectangle around the outline 206. While four QR codes are shown, it will be appreciated that all or many of the objectives herein may be met by the use of a different type of fiducial marker or a different number of fiducial markers. For example, the relative inclination of the test positioning card 200 with respect to a computing device 700 that is directed at the test positioning card 200 can be determined from an image of the test positioning card 200 (generated by the computing device 700) that includes three spaced-apart fiducial markers.

Since the fiducial markers 204 are of a known size and have a known relationship, the position of the test positioning card 200 (and hence of the test cassette 100, which is presumably positioned within the outline 206) with respect to the computing device 700 can be assessed from the size of each fiducial marker 204 in an image frame or from the distances between the fiducial markers 204 in the image frame. By checking that the distances between the fiducial markers, or the sizes of the fiducial markers, are within a certain tolerance, it can be determined that the test positioning card 200 is sufficiently close to the computing device 700 and sufficiently parallel to the plane of the image frame (or the plane of the image sensor being used to capture an image of the test positioning card 200).

The fiducial markers 204a to 204d may also be distinct from each other (e.g. include different data or information), which permits the orientation of the test positioning card 200 to be determined from the relative positions of the distinct fiducial markers 204 in an image frame. For example, if the fiducial marker 204a is detected in any relative position other than the top left in the image frame, appropriate steps may be taken, such as providing an output on a display of the computing device 700 prompting a user to rotate the card appropriately. Alternatively, if necessary (since ultimately it is the orientation of the test cassette 100 in the image frame that is important and not the orientation of the test positioning card 200), the computing device may automatically flip the acquired image before performing any analysis. Instead of or in addition to visual prompts or error messages as discussed herein, the computing device 700 may for example provide audible prompts or error messages.

Furthermore, since the fiducial markers 204 are in a known arrangement, the rotational alignment of test positioning card 200 to the image sensor or image frame can also be determined and appropriate steps taken if the test positioning card 200 is misaligned from the image sensor. That is, if the orientation of the test positioning card 200 is generally correct, with fiducial marker 204a in the top left position, but the test positioning card 200 is tilted to the left or the right in the image frame, an output can be provided on a display of the computing device 700 prompting a user to straighten the card.

As will be discussed in more detail below, the capture of a still image including the test cassette 100 occurs when the test positioning card 200 is positioned appropriately with respect to the computing device 700 and the test cassette 100 is appropriately positioned on the test positioning card 200. One of the control parameters is the distance of the test positioning card 200 from the computing device 700. If the test positioning card 200 is too close, the image frame may not be in focus, and if the test positioning card 200 is too far away, the part of the image frame corresponding to the test strip 108 may not be of sufficient resolution or quality so as to permit analysis thereof.

The spacing between the fiducial markers 204 on the test card is selected so that if the computing device 700 is too close to the test positioning card 200, not all of the fiducial markers 204 will appear in the image frame. In such a case, a user prompt may be generated instructing the user to move the computing device 700 further away from the test positioning card 200. In this regard, the aspect ratio of the rectangular arrangement of the fiducial markers 204 is chosen to correspond generally to the aspect ratio of the image frame, or to the aspect ratio of a smartphone display screen, or the aspect ratio of a display window in a smartphone app, e.g. 9:16 or 3:4 in portrait mode. By providing this general aspect ratio correspondence between the test positioning card 200 and the display of the image of the test positioning card on the computing device 700, a natural visual alignment cue is provided to a user. That is, it is logical for the user to position the computing device 700 such that the four fiducial markers 204 appear generally in the four corners of the display or display window of the computing device 700.

Whether or not the test positioning card 200 is too far away from the computing device 700 can be determined from the distance(s) between two or more fiducial markers 204, or from the size of one or more of the fiducial markers 204. If the distance between two identified fiducial markers 204 (e.g. fiducial marker 204a and fiducial marker 204b) is shorter than a predetermined threshold, indicating that the test positioning card is further from the computing device 700 than is preferred, a user prompt can be generated instructing the user to bring the computing device 700 closer to the test positioning card 200.

Similarly, if the distance between two identified fiducial markers 204 (e.g. fiducial marker 204a and fiducial marker 204b) is larger than a predetermined threshold, indicating that the computing device 700 is closer to the test positioning card 200 than is preferred, a user prompt can be generated instructing the user to move the computing device 700 further from the test position card 200.

The outline 206 provides a visual guide to the user for positioning and aligning the test cassette 100 on the test positioning card 200. As can be seen, the length and width of the outline 206 defines a generally rectangular shape that corresponds to the outline or perimeter of the test cassette 100, into which a user can place a test cassette 100. The computing device 700 can then perform test cassette recognition on the image frame to detect a test cassette 100 placed on the test positioning card 200, and the correct positioning and alignment of the test cassette 100 on the test positioning card 200 can be verified. If a test cassette is not detected, or the test cassette 100 is misaligned, appropriate warnings can be generated to alert the user.

Orientation of the test cassette 100 in the image frame can be determined using known image processing techniques. In one example, positioning of the test cassette 100 with respect to the test positioning card 200 is determined by computing the angle of a primary axis of the detected test cassette 100 with respect to a primary axis of the test positioning card 200, determined from the fiducial markers 204. In the event that the test cassette 100 is not correctly positioned or aligned within specified tolerances, a user prompt can be provided on a display screen of the computing device 700, instructing the user to align the test cassette 100 correctly on the test positioning card 200.

Whether or not the computing device 700 used to image the test positioning card 200 is oriented correctly, i.e. is relatively parallel to the test positioning test positioning card 200 and the test cassette 100, can also be determined by the location of the three or more fiducial markers 204 in the image stream observed by the computing device 700. For example, standard image processing and computer vision techniques to determine camera pose, and thereby computing device 700 pose, by determining a homography transform between the detected location of four fiducial markers and the known dimensions of the test positioning card 200 may be used. The determined pose of the camera or the computing device 700 with respect to the test positioning card 200 can then be used to guide the user to correctly position the computing device 700 to achieve appropriate frontal imaging of the test cassette 100.

In the illustrated example, the test positioning card 200 is white in color, which prevents the image of the test cassette 100 that is captured from being washed out or overexposed as a result of automatic exposure algorithms trying to compensate for a dark background. The computing device 700 normally has a flash or flashlight, which may be activated whenever a still image of the test cassette 100 is captured. This may be done to provide consistent lighting of the test cassette 100 as well as to reduce or eliminate any shadows that might be present in ambient lighting.

As shown, the test positioning card 200 may include various text or symbolic instructions, such as instructions 208 to “Scan the test with this side up” and instructions 210 to “Place the rapid test here.”

FIG. 3 is a flow chart illustrating a method of capturing a frontal image of a test cassette 100 that is positioned correctly on a test positioning card 200, in one example. Other variations of such a flowchart are feasible and can be easily implemented to satisfy different imaging constraints that may be imposed. The method commences after a user of a computing device has launched a software application for capturing diagnostic test result images and has selected an image capture option. The computing device in one example is a smartphone or other portable device with one or more image input components (e.g., one or more cameras) and a display, and the software application may comprise instructions executing on one or more processors of the computing device. An example of an appropriate computing device is the computing device 700 described below with reference to FIG. 7.

The software application typically provides a user interface on the display of the computing device 700, including an option to capture an image of diagnostic test results. Upon selection of the image capture option by the user, the software application will set any appropriate camera options, modes or lens selection. In one example, the software application will select a standard camera (if the computing device 700 has multiple cameras) and specify a standard mode with no zoom or other image enhancements, to try and ensure that images captured by different computing devices 700 are as uniform as possible. An example of a computing device 700 is the computing device 700 described below with reference to FIG. 7.

Upon activation of the image capture mode in the software application, a feed of images captured by an image input component is provided to the software application. The image feed typically comprises a sequence of image frames and in one example comprises a video stream received from the camera (or other image input component) of the computing device 700. The image feed is shown on the display of the computing device 700 to assist the user in aiming the computing device 700 at the test positioning card 200. At this point, the user will likely have positioned a test cassette 100 on a test positioning card 200 and pointed the camera of the computing device 700 at the test cassette 100. The method starts at operation 302 with the software application determining if a fiducial marker 204 (e.g. a QR code) is detected in the image feed and if this is one of the fiducial markers that is associated with the test positioning card 200. The software application will continue trying to detect a fiducial marker 204 associated with the test positioning card 200 until one is detected or until the user cancels the image capture mode. If an inappropriate fiducial marker is detected by the software application, an appropriate error message can be provided to the user.

Once an appropriate fiducial marker 204 is detected in operation 302, the method passes to operation 304 where the software application determines whether or not at least three fiducial markers 204 are found in the image feed. If less than three fiducial markers 204 are found, the software application displays (operation 306) an error message on a display of the computing device 700 that instructs a user to position the computing device 700 so that all the fiducial markers 204 on the test positioning card 200 are visible, or to move the computing device 700 further from the test positioning card 200. The software application then continues determining whether or not at least three fiducial markers 204 are found in the image feed in operation 304 until at least three fiducial markers 204 are detected or the user cancels the image capture mode.

Once the software application detects at least three appropriate fiducial markers 204 in operation 304, the method passes to operation 308 and operation 310 where the software application measures an imaging distance based on the size of one or more fiducial markers or on the distance(s) between two or more fiducial markers. Since the field of view of standard cameras included in portable computing devices such as smartphones tends to be fairly consistent, the imaging distance can be measured as a percentage or fraction of an image dimension. For example, the difference between they axis values of the locations of fiducial marker 204a and fiducial marker 204b in the image can be compared to the image height. If a fiducial marker 204 is too small or the distance between two fiducial markers 204 is too small compared to a predetermined threshold, the test in operation 310 fails and the software application displays an error message on the display of the computing device 700 at operation 312, indicating that the computing device 700 is too far from the test positioning card 200 and/or that the user needs to move the computing device 700 closer to the test positioning card 200.

It will be appreciated that determining that the test positioning card 200 is not too far away from the computing device 700 may be accomplished in many different ways, including for example determining an image distance between two fiducial markers using both x and y values of the locations of the respective fiducial markers, or by verifying that the test positioning card 200 is sufficiently aligned before making a determination along either the x or they dimension. For example, the alignment of the test positioning card 200 can be verified by comparing, for example, the y values of the locations of fiducial marker 204a and fiducial marker 204c and verifying that the difference between the two y values is below a certain threshold.

The particular thresholds for checking the positioning of the test positioning card 200 are not overly critical, are a matter of design choice, and acceptable values can readily be determined for a particular implementation by experimentation.

It is typically only necessary for the software application to check whether or not the computing device 700 is too far from the test positioning card 200, since if the computing device 700 is too close to the test positioning card 200, the test at operation 304 for at least three visible fiducial markers will fail. However, in other implementations, a check can also be included to restrict close-up imaging of the test cassette 100 as discussed above. The software application continues the measurement of operation 308 and determination of operation 310 until the computing device 700 is at an appropriate distance from the test positioning card 200 or the user cancels the image capture mode.

Once the software application has determined that the computing device 700 is at a sufficient distance in operation 310, the software application determines (at operation 314) the correspondence between the plane of the test positioning card 200 and the plane of the images in the image feed (i.e. the relative pose of the camera used to perform imaging). That is, the plane of the test positioning card is compared to the image plane or a relevant plane of the computing device 700. This is done by the software application comparing the distances between at least three of the fiducial markers 204 with each other, or by comparing the relative sizes of at least three of the fiducial markers 204 with each other, or by computing the angle formed between two sides of a quadrilateral shape (four fiducial markers detected) or triangle (three fiducial markers detected) formed by joining the detected locations of the fiducial markers in the imaging plane.

As for operation 310, the distances or sizes can be determined with respect to an image dimension. In one example, the pitch of the test positioning card 200 with respect to the computing device 700 can be assessed by comparing the horizontal distance between fiducial marker 204a and fiducial marker 204c and the horizontal distance between fiducial marker 204b and fiducial marker 204d. If the difference is greater than a predetermined threshold, the test positioning card 200 is not sufficiently parallel to the computing device 700. Similarly, the roll of the card can be assessed by comparing the distance between fiducial marker 204a and fiducial marker 204b and the vertical distance between fiducial marker 204c and fiducial marker 204d. If the difference is greater than a predetermined threshold, the test positioning card 200 is again not sufficiently parallel to the computing device 700. Also, the software application can determine the camera pose (roll, pitch and relative location) using the location of the fiducial markers 204 on the test positioning card 200 using standard image processing techniques.

If the computing device 700 and the test positioning card 200 are not sufficiently parallel as determined in operation 316 then the software application displays an error message on the display of the computing device 700 at operation 318, indicating that the computing device 700 is not parallel to the test positioning card 200.

The software application continues the measurement of operation 314 and determination of operation 316 until the computing device 700 is sufficiently parallel to the test positioning card 200 or the user cancels the image capture mode.

Once the software application has determined that the computing device 700 is sufficiently parallel in operation 316, the software application attempts to detect the presence of a test cassette 100 in the image frame(s) in operation 320. This is done by visual object recognition and comparison performed by the software application in a known manner or by a machine learning scheme that has been trained on an image set with identified test cassettes 100. If a test cassette 100 is not detected in operation 320 then the test in operation 322 fails and the software application displays an error message on the display of the computing device 700 at operation 324, indicating that a test cassette 100 is not present and prompting a user to place the test cassette 100 within the outline 206. The software application continues the attempted detection of a test cassette 100 in operation 320 and determination of operation 322 until the test cassette 100 is detected or the user cancels the image capture mode.

Once the software application has detected a test cassette 100 in the image frame(s) in in operation 322, the software application determines in operation 326 whether or not the detected test cassette 100 is positioned within boundaries defined with respect to the fiducial markers 204. This is done in one example by ensuring that each corner of the test cassette 100 (detected using corner detection image processing techniques) is within the outer corners of the fiducial markers 204, although it will be appreciated that different boundaries could be specified. In this regard, the outline 206 typically defines a stricter boundary than the boundary used by the software application in operation 326. This permits some variation in successful placement that is not strictly within the outline 206, which primarily serves as a visual guide for a user. If a test cassette 100 is not within the boundaries used in the test of operation 326 then the software application displays an error message on the display of the computing device 700 at operation 328, indicating that a test cassette 100 is incorrectly positioned, and prompting a user to place the test cassette 100 within the outline 206. The software application continues the attempted detection of a correctly positioned test cassette 100 in operation 326 until the test cassette 100 is positioned correctly or the user cancels the image capture mode.

After the software application has verified that the test cassette 100 is correctly located on the test positioning card 200 as in operation 326, the software application determines in operation 330 whether or not the detected test cassette 100 is vertically positioned. This is done in one example by comparing the angle of the test cassette 100 in the image frame, determined from the detected corners of the test cassette 100, with either the vertical axis of the test positioning card 200 or the y axis of the image frame. If the difference in the two angles is not within an acceptable tolerance, (for example +/−30 degrees) then the software application displays an error message on the display of the computing device 700 at operation 332, indicating that the test cassette 100 is not vertical and prompting a user to adjust or straighten the test cassette 100. The software application continues the attempted detection of a correctly aligned test cassette 100 in operation 330 until the test cassette 100 is positioned correctly or the user cancels the image capture mode.

Once the software application has detected that the test cassette 100 in the image is sufficiently vertical in operation 330, the software application then proceeds to an image capture step at operation 332, in which a still image is captured from the image feed. In one example, to reduce the possibility that the positions of the computing device 700, test positioning card 200 and test cassette 100 will be altered by requiring additional input or manipulation of the computing device 700 by the user, the software application may automatically capture the image once the requirements in the flowchart of FIG. 3 have been met. This may take place immediately, as soon as all the requirements of FIG. 3 have been met, or the software application may display a relevant message on the display for example, “Please hold still, image capture in . . . ” and a “3, 2, 1” countdown to image capture.

If the image is captured automatically, the software application may just examine the continual stream of video frames from the camera until it observes one that meets the acceptance criteria, and then use that frame instead of taking a photo or initiating a separate image capture step.

To facilitate reasonably consistent image capture across different computing devices 700 and under different circumstances, e.g. of ambient lighting, the software application typically enables a flash of the computing device 700 and but otherwise allows the computing device 700 to set the exposure for the capture of the image of the test cassette 100 and test positioning card 200. In one example, the flash of the computing device 700 is set (forced on) by the software application to go off at the time of the image capture. In another example, the flash/flashlight is illuminated constantly for some or all of the time, for example if the software application is going to capture a frame from the video feed automatically as soon as all of the requirements are met.

It will also be appreciated that while the determinations in FIG. 3 are described sequentially for purposes of clarity, the software application will continually monitor each of the conditions and if a condition that was satisfied earlier fails later, appropriate steps will be taken to rectify the situation. For example, if after satisfying the test in operation 310, the computing device 700 is then moved away from the test positioning card 200 so that the test in operation 310 fails, the error message of operation 312 will be displayed and the image will not be captured.

After a still image of the test cassette 100 positioned on the test positioning card 200 has been captured at operation 334, the method continues as shown in FIG. 5.

FIG. 4 is a flow chart illustrating a method of capturing a frontal image of a test cassette 100, in another example. Other variations of such a flowchart are feasible and can be easily implemented to satisfy different imaging constraints that may be imposed. The method commences after a user of a computing device has launched a software application for capturing diagnostic test result images and has selected an image capture option. The computing device in one example is a smartphone or other portable device with one or more image input components (e.g., one or more cameras) and a display, and the software application may comprise instructions executing on one or more processors of the computing device. An example of an appropriate computing device is the computing device 700 described below with reference to FIG. 7.

The software application typically provides a user interface on the display of the computing device 700, including an option to capture an image of diagnostic test results. Upon selection of the image capture option by the user, the software application will set any appropriate camera options, modes or lens selection. In one example, the software application will select a standard camera (if the computing device 700 has multiple cameras) and specify a standard mode with no zoom or other image enhancements, to try and ensure that images captured by different computing devices 700 are as uniform as possible. An example of a computing device is the computing device 700 described below with reference to FIG. 7.

Upon activation of the image capture mode in the software application, a feed of images captured by an image input component is provided to the software application. The image feed typically comprises a sequence of image frames and in one example comprises a video stream received from the camera (or other image input component) of the computing device 700. The image feed is shown on the display of the computing device 700 to assist the user in aiming the computing device 700 at a test cassette 100. The method starts at operation 402 with the software application determining if a test cassette 100 can be detected in the image feed. If a test cassette 100 is not detected by the software application at operation 404, an appropriate error message (e.g. “no test cassette detected”) can be provided to the user at operation 406.

The detection performed by the software application in operation 404 includes the detection or extraction of image features that are characteristic to the test cassette 100. For example, the edges/sides of the test cassette 100 or the corners of the test cassette 100 in the image frame may be detected using standard image processing techniques. Additionally, markings may be provided on the test cassette 100, including fiducial markers (if space permits) or other characteristic markings to enable the test cassette 100 to be detected and to allow its position in an image frame to be determined.

Once the software application has detected the test cassette 100 in operation 404, the method passes to operation 408 and operation 410 where the software application measures an imaging distance based on the image features of the test cassette 100 as extracted from the image frame. In one example, the distance in the image frame between two detected corners can be determined, to determine a height or width or diagonal measurement. Since the field of view of standard cameras included in portable computing devices such as smartphones tends to be fairly consistent, the imaging distance can be measured as a percentage or fraction of an image dimension.

If the measured distance is too large or too small compared to a predetermined threshold, the test in operation 410 fails and the software application displays an error message on the display of the computing device 700 at operation 412, indicating that the computing device 700 is either too near or too far from the test cassette 100 and/or that the user needs to move the computing device 700 further from or closer to the test cassette 100.

It will be appreciated that determining that the test cassette 100 is an appropriate distance from the computing device 700 may be accomplished in many different ways, including for example by determining an image distance between two or more corners using both x and y values of the locations of the corners in the image, or by verifying that the test cassette 100 is sufficiently aligned before making a determination along either the x or the y dimension. For example, the alignment of the test cassette 100 can be verified by comparing, for example, the y values of either the two lowest or the two highest corners in the image and verifying that the difference between the two y values is below a certain threshold, or by determining an angle of a detected edge of the test cassette 100.

The particular thresholds for checking the positioning of the test cassette 100 are not overly critical, are a matter of design choice, and acceptable values can readily be determined for a particular implementation by experimentation.

The software application continues the measurement of operation 408 and determination of operation 410 until the computing device 700 is at an appropriate distance from the test cassette 100 or the user cancels the image capture mode.

Once the software application has determined that the computing device 700 is at a sufficient distance in operation 410, the software application determines (at operation 414) the correspondence between the plane of the test cassette 100 and the plane of the images in the image feed (i.e. the relative pose of the camera used to perform imaging). That is, the plane of the test cassette 100 is compared to the image plane or a relevant plane of the computing device 700. This is done by the software application comparing the distances between at least three of the detected image features (e.g. corners) of the test cassette 100 with each other, or by computing the angle formed between two sides of a quadrilateral shape (four image features detected) or triangle (three image features detected) formed by joining the detected locations of the image features in the imaging plane.

As for operation 410, the distances can be determined with respect to an image dimension. In one example, the pitch of the test cassette 100 with respect to the computing device 700 can be assessed by comparing the distance between the two lower corners of the test cassette 100 with the distance between the two upper corners. If the difference is greater than a predetermined threshold, the test cassette 100 is not sufficiently parallel to the computing device 700. Similarly, the roll of the test cassette 100 can be assessed by comparing the distance between the two left corners of the test cassette 100 with the two right corners. If the difference is greater than a predetermined threshold, the test cassette 100 is again not sufficiently parallel to the computing device 700. Also, the software application can determine the camera pose (roll, pitch and relative location) using the location of image features on the test cassette 100 using standard image processing techniques.

If the computing device 700 and the test cassette 100 are not sufficiently parallel as determined in operation 416 then the software application displays an error message on the display of the computing device 700 at operation 418, indicating that the computing device 700 is not parallel to the test cassette 100.

The software application continues the measurement of operation 414 and determination of operation 416 until the computing device 700 is sufficiently parallel to the test cassette 100 or the user cancels the image capture mode.

Once the software application has determined that the computing device 700 is sufficiently parallel in operation 416, the software application determines in operation 420 whether or not the detected test cassette 100 is vertically positioned. This is done in one example by comparing the angle of the test cassette 100 in the image frame, determined from the detected corners of the test cassette 100, with the y axis of the image frame. If the difference is not within an acceptable tolerance, (for example +/−30 degrees) then the software application displays an error message on the display of the computing device 700 at operation 422, indicating that the test cassette 100 is not vertical and prompting a user to adjust or straighten the test cassette 100. The software application continues the attempted detection of a correctly aligned test cassette 100 in operation 420 and operation 422 until the test cassette 100 is positioned correctly or the user cancels the image capture mode.

Once the software application has detected that the test cassette 100 in the image is sufficiently vertical in operation 420, the software application then proceeds to an image capture step at operation 424, in which a still image is captured from the image feed. In one example, to reduce the possibility that the relative positions of the computing device 700 and test cassette 100 will be altered by requiring additional input or manipulation of the computing device 700 by the user, the software application may automatically capture the image once the requirements in the flowchart of FIG. 4 have been met. This may take place immediately, as soon as all the requirements of FIG. 4 have been met, or the software application may display a relevant message on the display for example, “Please hold still, image capture in . . . ” and a “3, 2, 1” countdown to image capture.

If the image is captured automatically, the software application may just examine the continual stream of video frames from the camera until it observes one that meets the acceptance criteria, and then use that frame instead of taking a photo or initiating a separate image capture step.

To facilitate reasonably consistent image capture across different computing devices 700 and under different circumstances, e.g. of ambient lighting, the software application typically enables a flash of the computing device 700 and but otherwise allows the computing device 700 to set the exposure for the capture of the image of the test cassette 100. In one example, the flash of the computing device 700 is set (forced on) by the software application to go off at the time of the image capture. In another example, the flash/flashlight is illuminated constantly for some or all of the time, for example if the software application is going to capture a frame from the video feed automatically as soon as all of the requirements are met.

It will also be appreciated that while the determinations in FIG. 4 are described sequentially for purposes of clarity, the software application will continually monitor each of the conditions and if a condition that was satisfied earlier fails later, appropriate steps will be taken to rectify the situation. For example, if after satisfying the test in operation 410, the computing device 700 is then moved away from the test cassette 100 so that the test in operation 410 fails, the error message of operation 412 will be displayed and the image will not be captured.

After a still image of the test cassette 100 has been captured at operation 424, the method continues as shown in FIG. 5.

FIG. 5 is a flow chart illustrating a method of verifying an image that has been captured by a computing device 700 using the method described above with reference to FIG. 3 or FIG. 4. One reason for performing the verification of FIG. 5 is that the user may have moved or be moving the computing device 700 after the tests in FIG. 3 or FIG. 4 have been satisfied but before the image is captured. The method of FIG. 5 is performed by the software application in one example, but may also be performed by a remote computing device after the captured image has been transmitted from the computing device 700 to the remote device for further analysis.

The method commences at operation 502 with the software application determining whether the dimensions and other parameters (e.g. the resolution) of the captured image are correct. This is an additional verification. Because of the checks that have been performed as described above with reference to FIG. 3 or FIG. 4, it is likely that the image parameters are within acceptable limits. If the image dimension and any other parameters are not correct, the software application displays an error message on the display of the computing device 700 at operation 504 and prompts the user to recapture the image. The software application may provide instructions to the user indicating how better to take the image, and may also return automatically, after a short delay, to operation 302 in FIG. 3 or operation 402 in FIG. 4, as appropriate.

If the image dimension is correct at operation 502, the method proceeds at operation 506 where the software application attempts to detect the sample well 104 of the test cassette 100 using known object recognition techniques or by a machine learning scheme that has been trained on an image set with identified sample wells. This includes determining the location and dimensions of the sample well 104 if detected. If the sample well 104 is not detected at operation 506, the software application displays an error message on the display of the computing device 700 at operation 508 and prompts the user to recapture the image. The software application may provide instructions to the user indicating how better to take the image, and may also return automatically, after a short delay, to operation 302 in FIG. 3 or operation 402 in FIG. 4, as appropriate.

If a sample well 104 is detected at operation 506, the method proceeds at operation 510 where the software application attempts to detect the result well 106 of the test cassette 100 using known object recognition techniques or by a machine learning scheme that has been trained on an image set with identified result wells. Once detected, the location of the result well 106 is known or a relevant location parameter can be determined. If the result well 106 is not detected at operation 510, the software application displays an error message on the display of the computing device 700 at operation 512 and prompts the user to recapture the image. The software application may provide instructions to the user indicating how better to take the image, and may also return automatically, after a short delay, to operation 302 in FIG. 3 or operation 402 in FIG. 4, as appropriate.

If a result well 106 is detected at operation 510, the vertical positions in the captured still image of the result well 106 and the sample well 104 are compared at operation 514. If the sample well 104 is above the result well 106 in the image, the image is flipped at operation 516 so that the result well 106 is above the sample well 104.

If the sample well 104 is below the result well 106 in the image, the method proceeds at operation 518 where the software application determines the height of the result well 106 and determines whether or not the height of the result well 106 is within acceptable limits. Because of the checks that have been performed as described above with reference to FIG. 3 or FIG. 4, it is likely that the result well height is within acceptable limits. This check thus serves as a further verification not only of the position of the test cassette 100 but also of the correct detection of the result well by the software application. If the height of the result well 106 is not within acceptable limits as determined at operation 518, the software application displays an error message on the display of the computing device 700 at operation 520 and prompts the user to recapture the image. The software application may provide instructions to the user indicating how better to take the image, and may also return automatically, after a short delay, to operation 302 in FIG. 3 or operation 402 in FIG. 4, as appropriate.

If an acceptable height for the result well 106 is verified at operation 518, the method proceeds at operation 522 where the software application checks if the captured image is underexposed, or if a relevant portion (e.g. the region of interest in the result well 106) is underexposed. Any suitable method of checking image exposure may be used, but in one example the RGB values of the pixels in the region of interest are compared to a predetermined threshold. For example, if more than a certain percentage of pixels (e.g. 30%) have RGB values below 60, then the region of interest is deemed to be underexposed.

If the comparison in operation 522 reveals that the captured image is underexposed, then the software application displays an error message on the display of the computing device 700 at operation 512 indicating that the image is underexposed and prompting the user to recapture the image. The software application may provide instructions to the user indicating how better to take the image, and may also return automatically, after a short delay, to operation 302 in FIG. 3 or operation 402 in FIG. 4, as appropriate.

If it is determined that the captured image is not underexposed at operation 522, the method proceeds at operation 524 where the software application checks if the captured image is overexposed. Any suitable method of checking image exposure may be used, but in one example the RGB values of the pixels in the region of interest are compared to a predetermined threshold. For example, if more than a certain percentage of pixels (e.g. 30%) have RGB values above 240, then the region of interest is deemed to be overexposed. If the comparison in operation 524 reveals that the captured image is overexposed, then the method proceeds to operation 526, where the software application attempts to detect a control line 110 in the captured image. This detection is performed in the region of interest of the detected result well 106 or of the test strip 108.

If a control line 110 is detected in operation 526, then the software application displays an error message on the display of the computing device 700 at operation 528 indicating that the captured image is overexposed and prompts the user to recapture the image. The software application may provide instructions to the user indicating how better to take the image, and may also return automatically, after a short delay, to operation 302 in FIG. 3 or operation 402 in FIG. 4, as appropriate.

If a control line 110 is not detected in operation 526, then the software application displays an error message on the display of the computing device 700 at operation 528 indicating that a control line 110 has not been detected. In such a case, the test cassette 100 may not have been exposed to a sample and the software application may prompt, at operation 530, the user to recapture the image or to confirm that the test cassette 100 has been used correctly before prompting the user to recapture the image. The software application may provide instructions to the user indicating how better to take the image, and may also return automatically, after a short delay, to operation 302 in FIG. 3 or operation 402 in FIG. 4, as appropriate.

If it is determined in operation 524 that the captured image is not overexposed, the method proceeds to operation 532, where the software application attempts to detect a control line 110 in the captured image. If a control line 110 is not detected in operation 532, then the software application displays an error message on the display of the computing device 700 at operation 528 indicating that a control line 110 has not been detected. In such a case, the test cassette 100 may not have been exposed to a sample and the software application may prompt the user to recapture the image or to confirm that the test cassette 100 has been used correctly before prompting the user to recapture the image. The software application may provide instructions to the user indicating how better to take the image, and may also return automatically, after a short delay, to operation 302 in FIG. 3 or operation 402 in FIG. 4, as appropriate.

If a control line 110 is detected at operation 532, the captured image is output at operation 534. In some cases, the captured image is processed and analyzed by the software application to interpret the test strip 108 using known techniques for analyzing and interpreting diagnostic test strips. In other cases, the captured image may be stored locally or remotely for later processing, or may be transmitted to a remote server for analysis. In other cases, the test strip 108 may be cropped from the captured image and similarly analyzed, stored or transmitted as appropriate or desired.

FIG. 6 is a block diagram 600 illustrating a software architecture 604, which can be installed on any one or more of the devices described herein. The software architecture 604 is supported by hardware such as a machine 602 that includes processors 620, memory 626, and I/O components 638. In this example, the software architecture 604 can be conceptualized as a stack of layers, where each layer provides a particular functionality. The software architecture 604 includes layers such as an operating system 612, libraries 610, frameworks 608, and applications 606. Operationally, the applications 606 invoke API calls 650 through the software stack and receive messages 652 in response to the API calls 650.

The operating system 612 manages hardware resources and provides common services. The operating system 612 includes, for example, a kernel 614, services 616, and drivers 622. The kernel 614 acts as an abstraction layer between the hardware and the other software layers. For example, the kernel 614 provides memory management, Processor management (e.g., scheduling), component management, networking, and security settings, among other functionality. The services 616 can provide other common services for the other software layers. The drivers 622 are responsible for controlling or interfacing with the underlying hardware. For instance, the drivers 622 can include display drivers, camera drivers, BLUETOOTH® or BLUETOOTH® Low Energy drivers, flash memory drivers, serial communication drivers (e.g., Universal Serial Bus (USB) drivers), WI-FED drivers, audio drivers, power management drivers, and so forth.

The libraries 610 provide a low-level common infrastructure used by the applications 606. The libraries 610 can include system libraries 618 (e.g., C standard library) that provide functions such as memory allocation functions, string manipulation functions, mathematic functions, and the like. In addition, the libraries 610 can include API libraries 624 such as media libraries (e.g., libraries to support presentation and manipulation of various media formats such as Moving Picture Experts Group-4 (MPEG4), Advanced Video Coding (H.264 or AVC), Moving Picture Experts Group Layer-3 (MP3), Advanced Audio Coding (AAC), Adaptive Multi-Rate (AMR) audio codec, Joint Photographic Experts Group (JPEG or JPG), or Portable Network Graphics (PNG)), graphics libraries (e.g., an OpenGL framework used to render in two dimensions (2D) and three dimensions (3D) in a graphic content on a display), database libraries (e.g., SQLite to provide various relational database functions), web libraries (e.g., WebKit to provide web browsing functionality), and the like. The libraries 610 can also include a wide variety of other libraries 628 to provide many other APIs to the applications 606.

The frameworks 608 provide a high-level common infrastructure that is used by the applications 606. For example, the frameworks 608 provide various graphical user interface (GUI) functions, high-level resource management, and high-level location services. The frameworks 608 can provide a broad spectrum of other APIs that can be used by the applications 606, some of which may be specific to a particular operating system or platform.

In an example embodiment, the applications 606 may include a home application 636, a contacts application 630, a browser application 632, a book reader application 634, a location application 642, a media application 644, a messaging application 646, a game application 648, and a broad assortment of other applications such as a third-party application 640. The applications 606 are programs that execute functions defined in the programs. Various programming languages can be employed to create one or more of the applications 606, structured in a variety of manners, such as object-oriented programming languages (e.g., Objective-C, Java, or C++) or procedural programming languages (e.g., C or assembly language). In a specific example, the third-party application 640 (e.g., an application developed using the ANDROID™ or IOS™ software development kit (SDK) by an entity other than the vendor of the particular platform) may be mobile software running on a mobile operating system such as IOS™, ANDROID™, WINDOWS® Phone, or another mobile operating system. In this example, the third-party application 640 can invoke the API calls 650 provided by the operating system 612 to facilitate functionality described herein.

FIG. 7 is a diagrammatic representation of the computing device 700 within which instructions 710 (e.g., software, a program, an application, an applet, an app, or other executable code) for causing the computing device 700 to perform any one or more of the methodologies discussed herein may be executed. For example, the instructions 710 may cause the computing device 700 to execute any one or more of the methods described herein. The instructions 710 transform the general, non-programmed computing device 700 into a particular computing device 700 programmed to carry out the described and illustrated functions in the manner described. The computing device 700 may operate as a standalone device or may be coupled (e.g., networked) to other machines. In a networked deployment, the computing device 700 may operate in the capacity of a server machine or a client machine in a server-client network environment, or as a peer machine in a peer-to-peer (or distributed) network environment. The computing device 700 may comprise, but not be limited to, a server computer, a client computer, a personal computer (PC), a tablet computer, a laptop computer, a netbook, a set-top box (STB), a PDA, an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 710, sequentially or otherwise, that specify actions to be taken by the computing device 700. Further, while only a single computing device 700 is illustrated, the term “machine” shall also be taken to include a collection of machines that individually or jointly execute the instructions 710 to perform any one or more of the methodologies discussed herein.

The computing device 700 may include Processors 704, memory 706, and I/O components 702, which may be configured to communicate with each other via a bus 740. In an example embodiment, the Processors 704 (e.g., a Central Processing Unit (CPU), a Reduced Instruction Set Computing (RISC) Processor, a Complex Instruction Set Computing (CISC) Processor, a Graphics Processing Unit (GPU), a Digital Signal Processor (DSP), an ASIC, a Radio-Frequency Integrated Circuit (RFIC), another Processor, or any suitable combination thereof) may include, for example, a Processor 708 and a Processor 712 that execute the instructions 710. The term “Processor” is intended to include multi-core processors that may comprise two or more independent processors (sometimes referred to as “cores”) that may execute instructions contemporaneously. Although FIG. 7 shows multiple Processors 704, the computing device 700 may include a single Processor with a single core, a single Processor with multiple cores (e.g., a multi-core Processor), multiple processors with a single core, multiple processors with multiples cores, or any combination thereof.

The memory 706 includes a main memory 714, a static memory 716, and a storage unit 718, both accessible to the processors 704 via the bus 740. The main memory 706, the static memory 716, and storage unit 718 store the instructions 710 embodying any one or more of the methodologies or functions described herein. The instructions 710 may also reside, completely or partially, within the main memory 714, within the static memory 716, within machine-readable medium 720 within the storage unit 718, within at least one of the processors 704 (e.g., within the processor's cache memory), or any suitable combination thereof, during execution thereof by the computing device 700.

The I/O components 702 may include a wide variety of components to receive input, provide output, produce output, transmit information, exchange information, capture measurements, and so on. The specific I/O components 702 that are included in a particular machine will depend on the type of machine. For example, portable machines such as mobile phones may include a touch input device or other such input mechanisms, while a headless server machine will likely not include such a touch input device. It will be appreciated that the I/O components 702 may include many other components that are not shown in FIG. 7. In various example embodiments, the I/O components 702 may include output components 726 and input components 728. The output components 726 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The input components 728 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or another pointing instrument), tactile input components (e.g., a physical button, a touch screen that provides location and/or force of touches or touch gestures, or other tactile input components), audio input components (e.g., a microphone), and the like.

In further example embodiments, the I/O components 702 may include biometric components 730, motion components 732, environmental components 734, or position components 736, among a wide array of other components. For example, the biometric components 730 include components to detect expressions (e.g., hand expressions, facial expressions, vocal expressions, body gestures, or eye-tracking), measure biosignals (e.g., blood pressure, heart rate, body temperature, perspiration, or brain waves), identify a person (e.g., voice identification, retinal identification, facial identification, fingerprint identification, or electroencephalogram-based identification), and the like. The motion components 732 include acceleration sensor components (e.g., accelerometer), gravitation sensor components, rotation sensor components (e.g., gyroscope). The environmental components 734 include, for example, one or cameras, illumination sensor components (e.g., photometer), temperature sensor components (e.g., one or more thermometers that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment. The position components 736 include location sensor components (e.g., a GPS receiver Component), altitude sensor components (e.g., altimeters or barometers that detect air pressure from which altitude may be derived), orientation sensor components (e.g., magnetometers), and the like.

Communication may be implemented using a wide variety of technologies. The I/O components 702 further include communication components 738 operable to couple the computing device 700 to a network 722 or devices 724 via respective coupling or connections. For example, the communication components 738 may include a network interface Component or another suitable device to interface with the network 722. In further examples, the communication components 738 may include wired communication components, wireless communication components, cellular communication components, Near Field Communication (NFC) components, Bluetooth® components (e.g., Bluetooth® Low Energy), Wi-Fi® components, and other communication components to provide communication via other modalities. The devices 724 may be another machine or any of a wide variety of peripheral devices (e.g., a peripheral device coupled via a USB).

Moreover, the communication components 738 may detect identifiers or include components operable to detect identifiers. For example, the communication components 738 may include Radio Frequency Identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph, MaxiCode, PDF417, Ultra Code, UCC RSS-2D bar code, and other optical codes), or acoustic detection components (e.g., microphones to identify tagged audio signals). In addition, a variety of information may be derived via the communication components 738, such as location via Internet Protocol (IP) geolocation, location via Wi-Fi® signal triangulation, location via detecting an NFC beacon signal that may indicate a particular location, and so forth.

The various memories (e.g., main memory 714, static memory 716, and/or memory of the processors 704) and/or storage unit 718 may store one or more sets of instructions and data structures (e.g., software) embodying or used by any one or more of the methodologies or functions described herein. These instructions (e.g., the instructions 710), when executed by processors 704, cause various operations to implement the disclosed embodiments.

The instructions 710 may be transmitted or received over the network 722, using a transmission medium, via a network interface device (e.g., a network interface Component included in the communication components 738) and using any one of several well-known transfer protocols (e.g., hypertext transfer protocol (HTTP)). Similarly, the instructions 710 may be transmitted or received using a transmission medium via a coupling (e.g., a peer-to-peer coupling) to the devices 724. A machine readable medium can comprise a transmission medium or a storage medium. A machine readable medium can comprise a non-transitory storage medium. A machine readable medium can comprise a transmission medium or a storage medium.

The following numbered examples are embodiments.
1. A method of capturing an image of diagnostic test results, performed by one or more processors, the method comprising:
acquiring an image of a diagnostic test positioning card using an image capture device;
determining, by the one or more processors, a presence of a plurality of fiducial images on the test positioning card;
determining, by the one or more processors, a presence of a diagnostic test result carrier placed on the test positioning card;
verifying, by the one or more processors, positioning of the test result carrier on the test positioning card; and
depending on verification of the positioning of the test result carrier on the test positioning card, capturing an image including the test result carrier.
2. The method of example 1, further comprising:
determining, by the one or more processors, that a plane of the test positioning card is sufficiently parallel to a plane of the image capture device prior to capturing the image of the test result carrier.
3. The method of example 1 or example 2, further comprising:
determining, by the one or more processors, a proximity related parameter from the plurality of fiducial images on the test positioning card; and
providing an error message depending on the determination of the proximity related parameter.
4. The method of any one of examples 1 to 3, further comprising:
determining, by the one or more processors, that less than a threshold number of the plurality of fiducial images are visible in the image of the test positioning card; and
providing an error message.
5. The method of any one of examples 1 to 4, further comprising:
determining, by the one or more processors, an alignment of the test result carrier on the test positioning card; and
providing an error message depending on the alignment of the test result carrier on the test positioning card.
6. The method of any one of examples 1 to 5, further comprising:
determining, by the one or more processors, a vertical alignment parameter of the test result carrier; and
providing an error message depending on the vertical alignment parameter of the test result carrier.
7. The method of any one of examples 1 to 6, further comprising:
determining, using the one or more processors, an exposure parameter for the image of the test result carrier; and
providing an error message depending on the exposure parameter.
8. The method of example 7, further comprising:
determining, by the one or more processors, that the exposure parameter exceeds a desired exposure parameter;
determining, by the one or more processors, that a control line is not present in the image of the test result carrier; and
providing an error message that a control line is not present in the image of the test result carrier.
9. The method of any one of examples 1 to 8, further comprising:
determining, by the one or more processors, that a control line is not present in the image of the test result carrier; and
providing an error message that a control line is not present in the image of the test result carrier.
10. The method of any one of examples 1 to 9, further comprising:
determining, by the one or more processors, a position of a sample well in the image of the test result carrier;
determining, by the one or more processors, a position of a result well in the image of the test result carrier;
determining, by the one or more processors, that the position of the sample well is above the position of the result well in the image of the test result carrier; and
flipping the image of the test result carrier.
11. A machine-readable medium comprising instructions that, when executed by one or more processors of a machine, cause the machine to perform operations comprising:
acquiring an image of a diagnostic test positioning card using an image capture device;
determining a presence of a plurality of fiducial images on the test positioning card;
determining a presence of a diagnostic test result carrier placed on the test positioning card;
determining positioning of the test result carrier on the test positioning card; and
depending on the positioning of the test result carrier on the test positioning card, capturing an image including the test result carrier.
12. The machine-readable medium of example 11, wherein the operations further comprise:
determining that a plane of the test positioning card is sufficiently parallel to a plane of the image capture device prior to capturing the image of the test result carrier.
13. The machine-readable medium of example 11 or example 12, wherein the operations further comprise:
determining an alignment of the test result carrier on the test positioning card; and
providing an error message depending on the alignment of the test result carrier on the test positioning card.
14. The machine-readable medium of any one of examples 11 to 13, wherein the operations further comprise:
determining, by the one or more processors, a proximity related parameter from the plurality of fiducial images on the test positioning card; and
providing an error message depending on the determination of the proximity related parameter.
15. The machine-readable medium of any one of examples 11 to 14, wherein the operations further comprise:
determining, using the one or more processors, an exposure parameter for the image of the test result carrier; and
providing an error message depending on the exposure parameter.
16. A system comprising:
one or more processors; and
one or more machine-readable mediums storing instructions that, when executed by the one or more processors, cause the system to perform operations comprising:
acquiring an image of a diagnostic test positioning card using an image capture device;
determining a presence of a plurality of fiducial images on the test positioning card;
determining a presence of a diagnostic test result carrier placed on the test positioning card;
determining positioning of the test result carrier on the test positioning card; and
depending on the positioning of the test result carrier on the test positioning card, capturing an image including the test result carrier.
17. The system of example 16, wherein the operations further comprise:
determining that a plane of the test positioning card is sufficiently parallel to a plane of the image capture device prior to capturing the image of the test result carrier.
18. The system of example 16 or example 17, wherein the operations further comprise:
determining that less than a threshold number of the plurality of fiducial images are visible in the image of the diagnostic test positioning card; and
providing an error message.
19. The system of any one of examples 16 to 18, wherein the operations further comprise:
determining that a control line is not present in the image of the test result carrier; and
providing an error message that a control line is not present in the image of the test result carrier.
20. The system of any one of examples 16 to 19, wherein the operations further comprise:
determining a position of a sample well in the image of the test result carrier;
determining a position of a result well in the image of the test result carrier;
determining that the position of the sample well is above the result well in the image of the test result carrier; and
flipping the image of the test result carrier.
21. A method of capturing an image of diagnostic test results, performed by one or more processors, the method comprising:
acquiring an image of a test result carrier using an image capture device;
determining, by the one or more processors, image features in the image of the test result carrier;
verifying, by the one or more processors, positioning of the test result carrier relative to the image capture device using the image features; and
depending on verification of the positioning of the test result carrier relative to the image capture device, capturing an image including the test result carrier.
22. The method of example 21, further comprising:
determining, by the one or more processors, that a plane of the test result carrier is sufficiently parallel to a plane of the image capture device prior to capturing the image of the test result carrier.
23. The method of example 21 or example 22, further comprising:
determining, by the one or more processors, a proximity related parameter from the image features of the test result carrier; and
providing an error message depending on the determination of the proximity related parameter.
24. The method of any one of examples 21 to 23, further comprising:
determining, by the one or more processors, an alignment of the test result carrier relative to the image capture device; and
providing an error message depending on the alignment of the test result carrier relative to the image capture device.
25. The method of example 24, wherein the determination of alignment of the test result carrier comprises:
determining an angular difference between a primary axis of the test result carrier and either a y axis or an x axis of the image.
26. The method of any one of examples 21 to 25, further comprising:
determining, using the one or more processors, an exposure parameter for the image of the test result carrier; and
providing an error message depending on the exposure parameter.
27. The method of example 26, further comprising:
determining, by the one or more processors, that the exposure parameter exceeds a desired exposure parameter;
determining, by the one or more processors, that a control line is not present in the image of the test result carrier; and
providing an error message that a control line is not present in the image of the test result carrier.
28. The method of any one of examples 21 to 27, further comprising:
determining, by the one or more processors, that a control line is not present in the image of the test result carrier; and
providing an error message that a control line is not present in the image of the test result carrier.
29. The method of any one of examples 21 to 28, further comprising:
determining, by the one or more processors, a position of a sample well in the image of the test result carrier;
determining, by the one or more processors, a position of a result well in the image of the test result carrier;
determining, by the one or more processors, that the position of the sample well is above the position of the result well in the image of the test result carrier; and
flipping the image of the test result carrier.
30. A machine-readable medium comprising instructions that, when executed by one or more processors of a machine, cause the machine to perform operations comprising:
acquiring an image of a test result carrier using an image capture device;
determining, by the one or more processors, image features in the image of the test result carrier;
verifying, by the one or more processors, positioning of the test result carrier relative to the image capture device using the image features; and
depending on verification of the positioning of the test result carrier relative to the image capture device, capturing an image including the test result carrier.
31. The machine-readable medium of example 30, wherein the operations further comprise:
determining, by the one or more processors, that a plane of the test result carrier is sufficiently parallel to a plane of the image capture device prior to capturing the image of the test result carrier.
32. The machine-readable medium of example 30 or example 31, wherein the operations further comprise:
determining, by the one or more processors, an alignment of the test result carrier relative to the image capture device; and
providing an error message depending on the alignment of the test result carrier relative to the image capture device.
33. The machine-readable medium of any one of examples 30 to 32, wherein the determination of alignment of the test result carrier comprises:
determining an angular difference between a primary axis of the test result carrier and either a y axis or an x axis of the image.
34. The machine-readable medium of any one of examples 30 to 33, wherein the operations further comprise:
determining, by the one or more processors, a proximity related parameter from the image features of the test result carrier; and
providing an error message depending on the determination of the proximity related parameter.
35. The machine-readable medium of any one of examples 30 to 34, wherein the operations further comprise:
determining, using the one or more processors, an exposure parameter for the image of the test result carrier; and
providing an error message depending on the exposure parameter.
36. A system comprising:
one or more processors; and
one or more machine-readable mediums storing instructions that, when executed by the one or more processors, cause the system to perform operations comprising:
acquiring an image of a test result carrier using an image capture device;
determining, by the one or more processors, image features in the image of the test result carrier;
verifying, by the one or more processors, positioning of the test result carrier relative to the image capture device using the image features; and
depending on verification of the positioning of the test result carrier relative to the image capture device, capturing an image including the test result carrier.
37. The system of example 36, wherein the operations further comprise:
determining, by the one or more processors, that a plane of the test result carrier is sufficiently parallel to a plane of the image capture device prior to capturing the image of the test result carrier.
38. The system of example 36 or example 37, wherein the operations further comprise:
determining, by the one or more processors, a proximity related parameter from the image features of the test result carrier; and
providing an error message depending on the determination of the proximity related parameter.
39. The system of any one of examples 36 to 38, wherein the operations further comprise:
determining that a control line is not present in the image of the test result carrier; and
providing an error message that a control line is not present in the image of the test result carrier.
40. The system of any one of examples 36 to 39, wherein the operations further comprise:
determining a position of a sample well in the image of the test result carrier;
determining a position of a result well in the image of the test result carrier;
determining that the position of the sample well is above the result well in the image of the test result carrier; and
flipping the image of the test result carrier.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Although an overview of the inventive subject matter has been described with reference to specific examples, various modifications and changes may be made to these examples without departing from the broader scope of examples of the present disclosure. Such examples of the inventive subject matter may be referred to herein, individually or collectively, by the term “invention” merely for convenience and without intending to voluntarily limit the scope of this application to any single disclosure or inventive concept if more than one is, in fact, disclosed.

The examples illustrated herein are described in sufficient detail to enable those skilled in the art to practice the teachings disclosed. Other examples may be used and derived therefrom, such that structural and logical substitutions and changes may be made without departing from the scope of this disclosure. The Detailed Description, therefore, is not to be taken in a limiting sense, and the scope of various examples is defined only by the appended claims, along with the full range of equivalents to which such claims are entitled.

As used herein, the term “or” may be construed in either an inclusive or exclusive sense. Moreover, plural instances may be provided for resources, operations, or structures described herein as a single instance. Additionally, boundaries between various resources, operations, modules, engines, and data stores are somewhat arbitrary, and particular operations are illustrated in a context of specific illustrative configurations. Other allocations of functionality are envisioned and may fall within a scope of various examples of the present disclosure. In general, structures and functionality presented as separate resources in the example configurations may be implemented as a combined structure or resource. Similarly, structures and functionality presented as a single resource may be implemented as separate resources. These and other variations, modifications, additions, and improvements fall within a scope of examples of the present disclosure as represented by the appended claims. The specification and drawings are, accordingly, to be regarded in an illustrative rather than a restrictive sense.

What is claimed is:

1. A method of capturing an image of diagnostic test results, performed by one or more processors, the method comprising:
acquiring an image of a diagnostic test positioning card using an image capture device;
determining, by the one or more processors, a presence of a plurality of fiducial images on the test positioning card;
determining, by the one or more processors, a presence of a diagnostic test result carrier placed on the test positioning card;
verifying, by the one or more processors, positioning of the test result carrier on the test positioning card; and
depending on verification of the positioning of the test result carrier on the test positioning card, capturing an image including the test result carrier.

2. The method of claim 1, further comprising:
determining, by the one or more processors, that a plane of the test positioning card is sufficiently parallel to a plane of the image capture device prior to capturing the image of the test result carrier.

3. The method of claim 1, further comprising:
determining, by the one or more processors, a proximity related parameter from the plurality of fiducial images on the test positioning card; and
providing an error message depending on the determination of the proximity related parameter.

4. The method of claim 1, further comprising:
determining, by the one or more processors, that less than a threshold number of the plurality of fiducial images are visible in the image of the test positioning card; and
providing an error message.

5. The method of claim 1, further comprising:
determining, by the one or more processors, an alignment of the test result carrier on the test positioning card; and
providing an error message depending on the alignment of the test result carrier on the test positioning card.

6. The method of claim 1, further comprising:
determining, by the one or more processors, a vertical alignment parameter of the test result carrier; and
providing an error message depending on the vertical alignment parameter of the test result carrier.

7. The method of claim 1, further comprising:
determining, using the one or more processors, an exposure parameter for the image of the test result carrier; and
providing an error message depending on the exposure parameter.

8. The method of claim 7, further comprising:
determining, by the one or more processors, that the exposure parameter exceeds a desired exposure parameter;
determining, by the one or more processors, that a control line is not present in the image of the test result carrier; and
providing an error message that a control line is not present in the image of the test result carrier.

9. The method of claim 1, further comprising:
determining, by the one or more processors, that a control line is not present in the image of the test result carrier; and
providing an error message that a control line is not present in the image of the test result carrier.

10. The method of claim 1, further comprising:
determining, by the one or more processors, a position of a sample well in the image of the test result carrier;
determining, by the one or more processors, a position of a result well in the image of the test result carrier;
determining, by the one or more processors, that the position of the sample well is above the position of the result well in the image of the test result carrier; and
flipping the image of the test result carrier.

11. A non-transitory machine-readable storage medium comprising instructions that, when executed by one or more processors of a machine, cause the machine to perform operations comprising:
acquiring an image of a diagnostic test positioning card using an image capture device;
determining a presence of a plurality of fiducial images on the test positioning card;
determining a presence of a diagnostic test result carrier placed on the test positioning card;
determining positioning of the test result carrier on the test positioning card; and
depending on the positioning of the test result carrier on the test positioning card, capturing an image including the test result carrier.

12. The non-transitory machine-readable storage medium of claim 11, wherein the operations further comprise:
determining that a plane of the test positioning card is sufficiently parallel to a plane of the image capture device prior to capturing the image of the test result carrier.

13. The non-transitory machine-readable storage medium of claim 11, wherein the operations further comprise:
determining an alignment of the test result carrier on the test positioning card; and
providing an error message depending on the alignment of the test result carrier on the test positioning card.

14. The non-transitory machine-readable storage medium of claim 11, wherein the operations further comprise:
determining, by the one or more processors, a proximity related parameter from the plurality of fiducial images on the test positioning card; and
providing an error message depending on the determination of the proximity related parameter.

15. The non-transitory machine-readable storage medium of claim 11, wherein the operations further comprise:
determining, using the one or more processors, an exposure parameter for the image of the test result carrier; and
providing an error message depending on the exposure parameter.

16. A system comprising:
one or more processors; and
one or more machine-readable mediums storing instructions that, when executed by the one or more processors, cause the system to perform operations comprising:
acquiring an image of a diagnostic test positioning card using an image capture device;
determining a presence of a plurality of fiducial images on the test positioning card;
determining a presence of a diagnostic test result carrier placed on the test positioning card;
determining positioning of the test result carrier on the test positioning card; and
depending on the positioning of the test result carrier on the test positioning card, capturing an image including the test result carrier.

17. The system of claim 16, wherein the operations further comprise:
determining that a plane of the test positioning card is sufficiently parallel to a plane of the image capture device prior to capturing the image of the test result carrier.

18. The system of claim 16, wherein the operations further comprise:
determining that less than a threshold number of the plurality of fiducial images are visible in the image of the diagnostic test positioning card; and
providing an error message.

19. The system of claim 16, wherein the operations further comprise:
determining that a control line is not present in the image of the test result carrier; and
providing an error message that a control line is not present in the image of the test result carrier.

20. The system of claim 16, wherein the operations further comprise:
determining a position of a sample well in the image of the test result carrier;
determining a position of a result well in the image of the test result carrier;
determining that the position of the sample well is above the result well in the image of the test result carrier; and
flipping the image of the test result carrier.

21. A method of capturing an image of diagnostic test results, performed by one or more processors, the method comprising:
acquiring an image of a test result carrier using an image capture device;
determining, by the one or more processors, image features in the image of the test result carrier;
verifying, by the one or more processors, positioning of the test result carrier relative to the image capture device using the image features; and
depending on verification of the positioning of the test result carrier relative to the image capture device, capturing an image including the test result carrier.

22. The method of claim 21, further comprising:
determining, by the one or more processors, that a plane of the test result carrier is sufficiently parallel to a plane of the image capture device prior to capturing the image of the test result carrier.

23. The method of claim 21, further comprising:
determining, by the one or more processors, a proximity related parameter from the image features of the test result carrier; and
providing an error message depending on the determination of the proximity related parameter.

24. The method of claim 21, further comprising:
determining, by the one or more processors, an alignment of the test result carrier relative to the image capture device; and
providing an error message depending on the alignment of the test result carrier relative to the image capture device.

25. The method of claim 24, wherein the determination of alignment of the test result carrier comprises:
determining an angular difference between a primary axis of the test result carrier and either a y axis or an x axis of the image.

26. The method of claim 21, further comprising:
determining, using the one or more processors, an exposure parameter for the image of the test result carrier; and
providing an error message depending on the exposure parameter.

27. The method of claim 26, further comprising:
determining, by the one or more processors, that the exposure parameter exceeds a desired exposure parameter;
determining, by the one or more processors, that a control line is not present in the image of the test result carrier; and
providing an error message that a control line is not present in the image of the test result carrier.

28. The method of claim 21, further comprising:
determining, by the one or more processors, that a control line is not present in the image of the test result carrier; and
providing an error message that a control line is not present in the image of the test result carrier.

29. The method of claim 21, further comprising:
determining, by the one or more processors, a position of a sample well in the image of the test result carrier;
determining, by the one or more processors, a position of a result well in the image of the test result carrier;
determining, by the one or more processors, that the position of the sample well is above the position of the result well in the image of the test result carrier; and
flipping the image of the test result carrier.

30. A non-transitory machine-readable storage medium comprising instructions that, when executed by one or more processors of a machine, cause the machine to perform operations comprising:
acquiring an image of a test result carrier using an image capture device;
determining, by the one or more processors, image features in the image of the test result carrier;
verifying, by the one or more processors, positioning of the test result carrier relative to the image capture device using the image features; and
depending on verification of the positioning of the test result carrier relative to the image capture device, capturing an image including the test result carrier.

31. The non-transitory machine-readable storage medium of claim 30, wherein the operations further comprise:
determining, by the one or more processors, that a plane of the test result carrier is sufficiently parallel to a plane of the image capture device prior to capturing the image of the test result carrier.

32. The non-transitory machine-readable storage medium of claim 30, wherein the operations further comprise:
determining, by the one or more processors, an alignment of the test result carrier relative to the image capture device; and
providing an error message depending on the alignment of the test result carrier relative to the image capture device.

33. The non-transitory machine-readable storage medium of claim 30, wherein the determination of alignment of the test result carrier comprises:
determining an angular difference between a primary axis of the test result carrier and either a y axis or an x axis of the image.

34. The non-transitory machine-readable storage medium of claim 30, wherein the operations further comprise:
determining, by the one or more processors, a proximity related parameter from the image features of the test result carrier; and
providing an error message depending on the determination of the proximity related parameter.

35. The non-transitory machine-readable storage medium of claim 30, wherein the operations further comprise:
determining, using the one or more processors, an exposure parameter for the image of the test result carrier; and
providing an error message depending on the exposure parameter.

36. A system comprising:
one or more processors; and
one or more machine-readable mediums storing instructions that, when executed by the one or more processors, cause the system to perform operations comprising:
acquiring an image of a test result carrier using an image capture device;
determining, by the one or more processors, image features in the image of the test result carrier;
verifying, by the one or more processors, positioning of the test result carrier relative to the image capture device using the image features; and
depending on verification of the positioning of the test result carrier relative to the image capture device, capturing an image including the test result carrier.

37. The system of claim 36, wherein the operations further comprise:
determining, by the one or more processors, that a plane of the test result carrier is sufficiently parallel to a plane of the image capture device prior to capturing the image of the test result carrier.

38. The system of claim 36, wherein the operations further comprise:
determining, by the one or more processors, a proximity related parameter from the image features of the test result carrier; and
providing an error message depending on the determination of the proximity related parameter.

39. The system of claim 36, wherein the operations further comprise:
determining that a control line is not present in the image of the test result carrier; and
providing an error message that a control line is not present in the image of the test result carrier.

40. The system of claim 36, wherein the operations further comprise:
determining a position of a sample well in the image of the test result carrier;
determining a position of a result well in the image of the test result carrier;
determining that the position of the sample well is above the result well in the image of the test result carrier; and
flipping the image of the test result carrier.
